(19) United States
(12) Patent Application Publication
(10) Pub. No.: US 2023/0263806 A1
(43) Pub. Date: Aug. 24, 2023

(54) METHODS OF TREATING PROTEINOPATHIES

(71) Applicant: Woolsey Pharmaceuticals, Inc., St. Petersburg, FL (US)

(72) Inventors: Thomas MACALLISTER, Arlington, VA (US); Sven JACOBSON, New York, NY (US)

(21) Appl. No.: 18/005,269

(22) PCT Filed: Jan. 8, 2021
(86) PCT No.: PCT/US21/12594

§ 371 Date: Jan. 12, 2023

Related U.S. Application Data
(60) Provisional application No. 63/051,532, filed on Jul. 14, 2020.

(51) Int. Cl. A61K 31/551

(52) U.S. Cl. CPC A61K 31/551

(57) ABSTRACT

The present invention relates to the treatment of conditions associated with a proteinopathy using a therapeutically effect amount of a rho kinase inhibitor. One preferred inhibitor is fasudil and preferred methods involve the daily oral administration of between 20 and 250 mg of fasudil. Proteinopathies preferably treated according to the invention involve aggregates of one or more of the following: amyloid beta, tau, Tar DNA Binding Protein 43 (TDP-43), Fused in sarcoma (FUS), α-synuclein, Huntingtin, Superoxide dismutase 1 (SOD-1), Prion proteins (PrP), mutant forms of Transthyretin, Atrophin 1 (ATN1), the Androgen receptor (AR), Ataxin 1 (ATXN1), Ataxin 2 (ATXN2), Ataxin 3 (ATXN3), Calcium Voltage-Gated Channel Subunit Alpha1 (ACACNA1A), Ataxin 7 (ATXN7), Protein Phosphatase 2 Regulatory Subunit Bbeta (PPP2R2B), and Tata Box Binding Protein (TBP).

### CROSS-REFERENCE TO RELATED APPLICATIONS

This application claims priority to U.S. provisional application No. 63/051,532, filed on Jul. 14, 2020, the disclosure of which is incorporated herein in its entirety.

### BACKGROUND OF THE INVENTION

Proteinopathies are a group of conditions resulting from the improper folding or unfolding of a protein. They typically, though not always, manifest as neurodegenerative disorders and are characterized by abnormal protein deposits or aggregates thought to result from conformational abnormalities that disrupt the normal placement of hydrophobic regions of the molecule into hydrophilic environments, causing the proteins to become insoluble and/or aggregate. In some cases, such as in prion diseases, the abnormal structures are thought to form nuclei for further protein deposition, initiating a kind of chain reaction of protein aggregation. The culprit proteins often are normal proteins with aggregates being formed after some post-translational modification, such as phosphorylation. One example of this is the group of disorders known as tauopathies, which result in deposits of the microtubule associated protein tau.

Examples of tauopathies include primary age-related tauopathy (PART)/Neurofibrillary tangle-predominant senile dementia, characterized by the presence of tau NFTs similar to AD, but without amyloid beta plaques; Lytico-bodig disease (Parkinson-dementia complex of Guam); pure autonomic failure associated with alpha-synuclein or other proteinopathies, ganglioglioma and gangliocytoma; meningioangiomatosis; postencephalitic parkinsonism; subacute sclerosing panencephalitis (SSPE) and argyrophilic grain disease (AGD). Infant tauopathies include hemimegalencephaly (HME), tuberous sclerosis complex, focal cortical dysplasia type 2b (FCD2b), and ganglioglioma, a tumor with dysplastic neurons and neoplastic glia cells. In other cases, the proteins are modified in some way that causes them to misfold and aggregate. An example of this is the huntingtin protein, which forms aggregates in Huntington's disease (HD) due to an amplification of a region encoding the amino acid glutamine. HD is an example of so-called poly-Q proteinopathies, due to the fact that that single letter code for glutamine is the letter Q.

Another example of a proteinopathy is prion diseases. Prions are made up of PrP, a membrane-bound protein of unknown function that is found throughout the body, even in healthy people. The normal form of the protein is called PrPC, while the infectious form is called PrPSc. The infectious form adopts different, abnormal structures that render it resistant to proteases. These misfolded forms of PrP have the ability to transmit their misfolded shape onto normal variants of the same protein. These form abnormal protein aggregates that accumulate in infected tissue and are associated with tissue damage and cell death. Similarly, amyloid beta in Alzheimer's Disease (AD) is thought to nucleate and propagate its aggregation into amyloid plaques in a matter similar to the way prion proteins propagate their own aggregation.

Chronic traumatic encephalopathy (CTE), a neurodegenerative disease caused by repeated head injuries, is a tauopathy characterized by the deposition of hyperphosphorylated tau (p-tau) protein as neurofibrillary tangles, astrocytic tangles and neurites in striking clusters around small blood vessels of the cortex, typically at the sulcal depths. One form of CTE is dementia pugilistica.

Autism spectrum disorder, Down syndrome, progressive supranuclear palsy, corticobasal degeneration, and various forms of dementia often are pathologically linked to a proteinopathy—a aggregates or deposits usually consisting of mis-folded proteins. The following table illustrates examples of proteinopathies and the principle proteins making up the abnormal aggregates.

| Proteinopathy | Aggregating protein(s) |
|---|---|
| Alzheimer's disease | Amyloid beta (Ab) peptide; |
| | Tau |
| Pick's Disease/Frontotemporal | Tau (microtubule associated) |
| Dementia | |
| Corticobasilar Degeneration | Tau (microtubule associated) |
| Progressive Supranuclear Palsy | Tau (microtubule associated) |
| Primary age-related tauopathy | Tau |
| (PART)/Neurofibrillary tangle- | |
| predominant senile dementia | |
| Infantile tauopathies | Tau (microtubule associated) |
| (hemimegalencephaly (HME), | |
| tuberous sclerosis complex, | |
| focal cortical dysplasia | |
| type 2b (FCD2b), | |
| ganglioglioma) | |
| Lytico-bodig disease (Parkinson- | Tau |
| dementia complex of Guam) | |
| ganglioglioma and gangliocytoma | Tau (NFT) |
| meningioangiomatosis | Tau |
| postencephalitic parkinsonism | Tau |
| subacute sclerosing panencephalitis | Tau |
| (SSPE) | |
| argyrophilic grain disease (AGD) | Tau |
| Frontotemporal Lobar Degeneration - | Tar DNA Binding Protein 43 |
| TDP | (TDP-43) |
| Limbic-predominant age-related | TDP-43 |
| TDP-43 encephalopathy (LATE), | |
| LATE-NC (neuropathological | TDP-43 |
| change) | |
| Perry Syndrome | Dynactin |
| Frontotemporal Lobar Degeneration - | Fused in sarcoma (FUS) |
| FUS | |
| Multiple system atrophy | α-synuclein |
| Parkinson's disease | α-synuclein |
| Lewy Body Disease | α-synuclein; TDP-43 |
| Pure autonomic failure | α-synuclein |
| Huntington's disease | Huntingtin with tandem |
| | glutamine repeats |
| Amyotrophic lateral sclerosis | Superoxide dismutase 1; |
| | TDP43 |
| Spongiform encephalopathies | Prion proteins |
| Familial amyloidotic polyneuropathy | Transthyretin (mutant forms) |
| Multisystem proteinopathy; associated | TDP-43 and other proteins |
| with valosin-containing protein -and | |
| mutations in prion-like domains of | |
| RNA binding proteins | |
| Retinitis pigmentosa | Rhodopsin |
| Inclusion body myopathy (IBM) | Mutant valosin-containing |
| associated with Paget's disease of the | protein (VCP) |
| bone (PDB), fronto-temporal dementia | |
| (FTD) and amyotrophic lateral | |
| sclerosis (ALS)—sometimes called | |
| IBMPFD/ALS | |
| UMOD-associated kidney disease | Mutant uromodulin protein |
| MUC1- associated kidney disease | Mutant MUC-1 |
| Respiratory Epithelial Cell | α1-antitrypsin and other |
| Proteinopathy (caused by surfactant | proteins |
| protein A and C deficiencies) | |
| Familial amyloidotic cardiomyopathy | Transthyretin |
| Familial amyloidotic poly-neuropathy | Mutant transthyretin |
| (FAP) | |
| Senile systemic amyloidosis | Transthyretin |
| Systemic amyloidosis (as a | Immunoglobulin light-chain |
| complication of multiple myeloma) | or light chain fragment |

A central issue with interventions that target proteinopathies is that of association versus causation. In order for an intervention to work in treating a disease, it must interrupt the chain of causation. AD, the most common form of dementia and a prototypical proteinopathy, provides a very instructive case. Like many proteinopathies, even many of those with systemic protein aggregates, AD affects the central nervous system. The two characteristic pathological findings of AD are the extracellular amyloid plaques and inter-neuronal neurofibrillary tangles (NFT), two different types of protein aggregates.

While Aβ, tau and neuroinflammation are certainly associated with AD, is it not clear they are involved in causation and thus, it is unclear that affecting any of these will have any therapeutic benefit in treating the disease. Based on understanding the familial disease, it is believed that Aβ starts the process of neurodegeneration by inducing Tau pathology, neuroinflammation and finally the neuronal loss that leads to cognitive decline. In other words, Aβ is at the beginning of the causality chain. Stopping Aβ pathology should stop the disease and, so far, most therapeutic approaches have targeted Aβ.

Despite the overwhelming literature showing the promise of targeting Aβ in animal models, however, there have been no products that have been shown to work in AD. Ceyzériat et al., 2020). These failures include, notably among many, Anti-Aβ42+Freud's adjuvant, Bapineuzumab, Solanezumab, Aducanumab, Verubecestat, Lanabecestat, Atabecestat, CNP520, Elenbecestat, γ-Secretase inhibitors, Bryostatin and PBT2.

Tau is a less likely target because of the evidence that it is downstream of Aβ, and thus is not causative, and so trials have been less frequent. Notably, of 15 trial targeting tau that have been initiated, already four of them have been stopped.

The role of neuroinflammation, the third putative interventional target, in AD is unclear, likely being beneficial in early-stage disease, but possibly evolving to a bad actor by participating in a loop of pro-inflammatory cytokine production and oxidative stress. While epidemiological studies have suggested that treatment with nonsteroidal anti-inflammatory drugs (NSAIDs) reduce the risk of developing AD and they can decrease amyloid load in transgenic models, to date prospective studies testing anti-inflammatory drugs have shown no beneficial effect on cognition in AD. Studies targeting neuroinflammation are ongoing, but early results are not promising. Neflamapimod, a selective inhibitor of p38 mitogen-activated protein kinase showed efficacy in an animal model, but it had no effect on Aβ deposition in humans and failed its primary endpoint of improving episodic memory in Phase 2, despite reducing tau in the cerebrospinal fluid.

In view of the number of clinical failures of compounds that seemed promising in animal models, a grave degree of skepticism should be applied in interpreting animal data. Even aside from the obvious issues of differences in brain complexity between rodents and humans, many of the existing models bear only a passing resemblance to the human condition. Many things can cause neural degeneration in animals and many putative drugs can halt that neural degeneration, but the underlying pathophysiology and chain of causation is unknown and it is there that a disease modifying intervention must act. It is crucial, therefore, that animal models, with their known deficiencies in the best of cases, as closely resemble the human disease as possible, in both pathology and clinical presentation.

There are a number of publications looking at the use of rho kinase inhibitors in various models of AD/dementia. Most models are deficient in basic properties. Some models involve the direct induction of neurotoxicity with agents like streptozotocin or even by direct injection of amyloid-beta into the brain. While these models may exhibit certain AD-like properties, they are basically just models of neural degeneration and cannot predict treatment of AD itself. Even the transgenic models are deficient. For example, there are a number of transgenic mice that only develop amyloid plaques without NFTs, such as the APP/PS-1 mouse, perhaps the most widely reported transgenic model. There are also mice that develop tauopathies, without amyloid plaques, such as the rTG4510 tau mouse. AD is characterized by the presence of both. Some publications use unrealistic routes of administration (e.g., intraventricular injection) and many do not use appropriate dosing. In this regard, standard formulas exist for converting doses used in animals to the same dose in humans. Human equivalent dose (HED) can be calculated, for example, using Table 1 of Nair & Jacob (2016), which are the same conversions used by the US FDA. Becker2008) discusses the criticality of dose in successful AD drug development and points to it as a failure point in AD drug development.

Published literature exists in which fasudil is administered in animal models of dementia. But these studies are deficient for many of the same reasons. Namely, the animal models do not faithfully recapitulate human disease, partly due to species differences in neuroanatomy (Sasaguri 2017) and partly due to the deficient basic pathological bases of the models, described above. In addition, some fail to use physiologically relevant doses and, importantly, no relevant outcomes. It is important also to note that the hallmark of onset in the paradigmatic cortical dementia, AD, is the failure of semantic memory, which cannot be measured in any animal model and so all animal models share this deficiency as well. For example, Hamano et al., 2019, administered 12 mg/kg/day (68 mg HED) to rTG4510 tau transgenic mice and measured only tau phosphorylation/cleavage and oligomers, but no outcomes. Elliott 2018 used a triple transgenic mouse model (APP Swedish, MAPT P301L, and PSEN1 M146V) and observed reduce 1 amyloid plaques in vivo at a dose of 10 mg/kg/day (intraperitoneally) fasudil (57 mg HED).

Sellers 2018 used the AB42 mouse model and administered fasudil intraperitoneally at a dose of 10 mg/kg BID (226 m HED) but monitored only ß-amyloid dendritic spine loss. Couch et al. 2010 used intraventricular infusion and observed effects on dendritic branching and no relevant outcomes. Putting aside the absence of any behavioral outcomes in these references, intraventricular administration is not a therapeutic option for humans. Yu 2017 and Hou 2012 administered fasudil at 5 and 10 nag/kg/day intraperitoneally to APP/PS1 transgenic mice (70, 140 mg HED) and streptozotocin rats (226 mg HED). respectively. Neither of these purported models recapitulates AD. As explained above, the APP/P1 model has no tau, which is the pathogenic actor. The rat model is a model of neurotoxicity, not AD, where a toxin (streptozotocin) is injected. directly into the brain, which is not physiologically relevant to the progressive neurodegeneration of AD.

Conflicting reports to the above also exist. For example, Turk 2018 (dissertation) used triple transgenic Mice and did not observe improvements in spatial memory at 10 or 12 months of age with fasudil administered in water at 30 mg/kg and 100 mg/kg.

Based on currently available animal modeling, different therapeutic strategies targeting the pathological hallmarks of dementia have been tested but have failed to show any beneficial effects in humans. At present, available medications are limited to acetylcholinesterase inhibitors and N-methyl-D-aspartate (NMDA) receptor antagonists, which show only modest improvements in some cognitive symptoms.

It has also been suggested in the literature that rho kinases inhibit autophagy and, thus, rho kinase inhibitors enhance autophagy; however, Gurkar 2013 showed that ROCK1 is a critical actor in autophagosome assembly and that inhibition of ROCK1 reduced autophagy, an observation supported by Hu 2016. Similar suggestions can be found regarding proteasome-mediated degradation, but Peng 2016 report that ROCK1 is a facilitator of proteasome-mediated protein degradation in wasting associate with chronic kidney disease and that ROCK1 knock-outs have blunted proteasomal activity.

There exists a significant unmet need to provide new, therapies that show benefit in humans, not just animals.

### SUMMARY OF THE INVENTION

The invention contemplates the treatment of conditions associated with an underlying proteinopathy with rho kinase inhibitors. In a preferred embodiment, the rho kinase inhibitor is fasudil and it is administered orally in a daily dose of between 70 and 250 mg per day.

Certain preferred embodiments treat conditions with proteinopathies of one or more of the following proteins: amyloid beta, tau, Tar DNA Binding Protein 43 (TDP-43), Fused in sarcoma (FUS), α-synuclein, Huntingtin, Superoxide dismutase 1 (SOD-1), Prion proteins (PrP), mutant forms of Transthyretin (TTR), Atrophin 1 (ATN1; aka DRPLA), the Androgen receptor (AR), Ataxin 1 (ATXN1), Ataxin 2 (ATXN2), Ataxin 3 (ATXN3), Calcium Voltage-Gated Channel Subunit Alpha1 (ACACNA1A), Ataxin 7 (ATXN7), Protein Phosphatase 2 Regulatory Subunit Bbeta (PPP2R2B), dynactin, rhodopsin, valosin-containing protein, uromodulin, MUC-1, α1-antitrypsin, immunoglobulin light chain, an immunoglobulin light chain fragment, and Tata Box Binding Protein (TBP). Other embodiments contemplate the treatment of diseases selected from the group consisting of Huntington's disease, autism spectrum disorder, Down syndrome (often associated with TDP-43), Alzheimer's Disease (AD), Dementia with Lewy Bodies (DLB), Frontotemporal Dementia (FTD, aka, Pick's Disease), and other forms of Frontotemporal Lobar Degeneration), head injuries, normal pressure hydrocephalus, Creutzfeldt-Jakob disease and other spongiform encephalopathies including Kuru, Gerstmann-Straussler-Scheinker syndrome, and fatal familial insomnia), amyotrophic lateral sclerosis, Corticobasilar Degeneration, Progressive Supranuclear Palsy, Frontotemporal Lobar Degeneration—TDP, limbic-predominant age-related TDP4-3 encephalopathy (LATE), with or without coexisting hippocampal sclerosis pathology, LATE-NC (atrophy focused in amygdala, hippocampus, and middle frontal gyrus), Frontotemporal Lobar Degeneration—FUS, Multiple system atrophy, Familial amyloidotic polyneuropathy, Dentatorubropallidoluysian atrophy, Spinal and bulbar muscular atrophy, Spinocerebellar ataxias (including Types 1, 2, 3, 6, 7, 12 and 17), Machado-Joseph disease, Pure Autonomic Failure, Parkinson's disease, multisystem proteinopathy retinitis pigmentosa, inclusion body myopathy (IBM) associated with Paget's disease of the bone (PDB), fronto-temporal dementia (FTD) and amyotrophic lateral sclerosis (ALS) [sometimes called IBMPFD/ALS], UMOD-associated kidney disease, MUC1-associated kidney disease, respiratory epithelial cell proteinopathy, familial amyloidotic cardiomyopathy, familial amyloidotic poly-neuropathy (FAP), senile systemic amyloidosis, and systemic amyloidosis as a complication of multiple myeloma.

In a further embodiment, the disease treated has combinations of the foregoing proteinopathies.

### DETAILED DESCRIPTION OF THE INVENTION

The invention is based on the discovery that rho kinase inhibitors can be used to treat disorders associated with an underlying proteinopathy. Proteinopathies underly many neurological diseases, including many forms of dementia. The invention, therefore, contemplates treating degenerative neurological conditions with underlying proteinopathies, such as Huntington's disease (HD), Parkinson's disease (PD), autism spectrum disorder, Down syndrome, multiple system atrophy, and dementia (for example Alzheimer's disease, frontotemporal dementia, dementia with Lewy Bodies, Creutzfeldt-Jakob dementia (CJD), normal pressure hydrocephalus, HD dementia and PD dementia).

ROCK Inhibitors

The inventive methods contemplate the administration of a rho kinase (ROCK) inhibitor in the treatment of a disease or condition. Two mammalian ROCK homologs are known, ROCK1 (aka ROKβ, Rho-kinase β, or p160ROCK) and ROCK2 (aka ROKα) (Nakagawa 1996). In humans, the genes for both ROCK1 and ROCK2 are located on chromosome 18. The two ROCK isoforms share 64% identity in their primary amino acid sequence, whereas the homology in the kinase domain is even higher (92%) (Jacobs 2006; Yamaguchi 2006). Both ROCK isoforms are serine/threonine kinases and have a similar structure.

A large number of pharmacological ROCK inhibitors are known (Feng, LoGrasso, Defert, & Li, 2015). Isoquinoline derivatives are a preferred class of ROCK inhibitors. The isoquinoline derivative fasudil was the first small molecule ROCK inhibitor developed by Asahi Chemical Industry (Tokyo, Japan). The characteristic chemical structure of fasudil consists of an isoquinoline ring, connected via a sulphonyl group to a homopiperazine ring. Fasudil is a potent inhibitor of both ROCK isoforms. In vivo, fasudil is subjected to hepatic metabolism to its active metabolite hydroxyfasudil (aka, M3). Other examples of isoquinoline derived ROCK inhibitors include dimethylfasudil and ripasudil.

Other preferred ROCK inhibitors are based on based on 4-aminopyridine structures. These were first developed by Yoshitomi Pharmaceutical (Uehata et al., 1997) and are exemplified by Y-27632. Still other preferred ROCK inhibitors incude indazole, pyrimidine, pyrrolopyridine, pyrazole, benzimidazole, benzothiazole, benzathiophene, benzamide, aminofurazane, quinazoline, and boron derivatives (Feng et al., 2015). Some exemplary ROCK inhibitors are shown below:

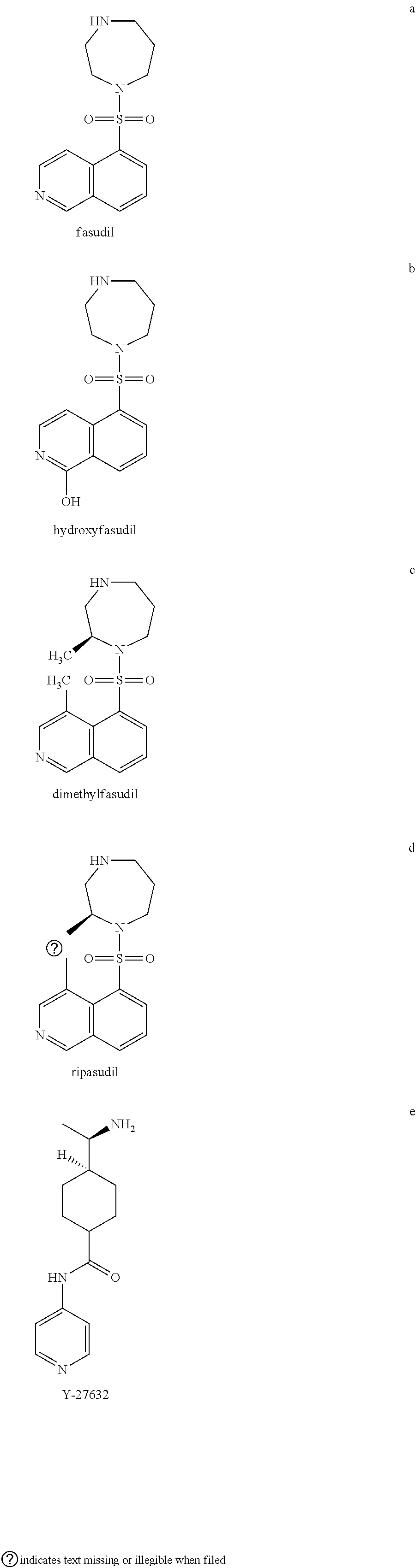

ROCK inhibitors according to the invention may have more selective activity for either ROCK1 or ROCK2 and will usually have varying levels of activity on PKA, PKG, PKC, and MLCK. Some ROCK inhibitors may be highly specific for ROCK1 or ROCK2 and have much lower activity against PKA, PKG, PKC, and MLCK.

A particularly preferred ROCK inhibitor is fasudil. Fasudil may exist as a free base or salt and may be in the form of a hydrate, such as a hemihydrate.

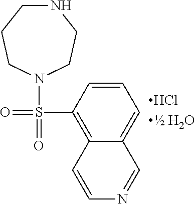 [SMILES: O=S(=O)(c1cccc2cnccc12)N1CCCNCC1]

### Hexahydro-1-(5-isoquinolinesulfonyl)-1H-1,4-diazepine Monohydrochloride Hemihydrate

Fasudil is a selective inhibitor of protein kinases, such as ROCK, PKC and MLCK and treatment results in a potent relaxation of vascular smooth muscle, resulting in enhanced blood flow (Shibuya 2001). A particularly important mediator of vasospasm, ROCK induces vasoconstriction by phosphorylating the myosin-binding subunit of myosin light chain (MLC) phosphatase, thus decreasing MLC phosphatase activity and enhancing vascular smooth muscle contraction. Moreover, there is evidence that fasudil increases endothelial nitric oxide synthase (eNOS) expression by stabilizing eNOS mRNA, which contributes to an increase in the level of the potent vasodilator nitric oxide (NO), thereby enhancing vasodilation (Chen 2013).

Fasudil has a short half-life of about 25 minutes, but it is substantially converted in vivo to its 1-hydroxy (M3) metabolite. M3 has similar effects to its fasudil parent molecule, with slightly enhanced activity and a half-life of about 8 hours (Shibuya 2001). Thus, M3 is likely responsible for the bulk of the in vivo pharmacological activity of the molecule. M3 exists as two tautomers, depicted below:

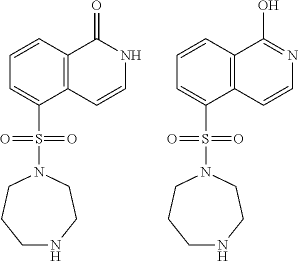 [SMILES: O=c1[nH]ccc2c(S(=O)(=O)N3CCCNCC3)cccc12 ; O=S(=O)(c1cccc2c(O)nccc12)N1CCCNCC1]

The ROCK inhibitors used in the invention, such as fasudil, include pharmaceutically acceptable salts and hydrates. Salts that may be formed via reaction with inorganic and organic acid. Those inorganic and organic acids are included as following: hydrochloric acid, hydrobromide acid, hydriodic acid, sulphuric acid, nitric acid, phosphoric acid, acetic acid, maleic acid, maleic acid, maleic acid, oxalic acid, oxalic acid, tartaric acid, malic acid, mandelic acid, trifluoroacetic acid, pantothenic acid, methane sulfonic acid, or para-toluenesulfonic acid.

Pharmaceutical Compositions

Pharmaceutical compositions of ROCK inhibitors usable in the are generally oral and may be in the form of tablets or capsules and may be immediate-release formulations or may be controlled- or extended-release formulations, which may contain pharmaceutically acceptable excipients, such as corn starch, mannitol, povidone, magnesium stearate, talc, cellulose, methylcellulose, carboxymethylcellulose and similar substances. A pharmaceutical composition comprising a ROCK inhibitor and/or a salt thereof may comprise one or more pharmaceutically acceptable excipients, which are known in the art. Formulations include oral films, orally disintegrating tablets, effervescent tablets and granules or beads that can be sprinkled on food or mixed with liquid as a slurry or poured directly into the mouth to be washed down.

Pharmaceutical compositions containing ROCK inhibitors, salts and hydrates thereof can be prepared by any method known in the art of pharmaceutics. In general, such preparatory methods include the steps of bringing a ROCK inhibitor or a pharmaceutically acceptable salt thereof into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. The composition used in accordance with the methods of the present invention may comprise between 0.001% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

In certain embodiments, the pharmaceutical composition used in the methods of the present invention may comprise a diluent. Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

In certain embodiments, the pharmaceutical composition used in the methods of the present invention may comprise a granulating and/or dispersing agent. Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and mixtures thereof.

In certain embodiments, the pharmaceutical composition used in the methods of the present invention may comprise a binding agent. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan), alginates, polyethylene oxide, polyethylene glycol, inorganic calcium salts, silicic acid, polymethacrylates, waxes, water, alcohol, and/or mixtures thereof.

In certain embodiments, the pharmaceutical composition used in the methods of the present invention may comprise a preservative. Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

In certain embodiments, the pharmaceutical composition used in the methods of the present invention may comprise an antioxidant. Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

In certain embodiments, the pharmaceutical composition used in the methods of the present invention may comprise a chelating agent. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

In certain embodiments, the pharmaceutical composition may comprise a buffering agent together with the ROCK inhibitor or the salt thereof. Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

In certain embodiments, the pharmaceutical composition used in the methods of the present invention may comprise a lubricating agent. Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

In other embodiments, the pharmaceutical composition of containing a ROCK inhibitor or salt thereof will be administered as a liquid dosage form. Liquid dosage forms for oral and parenteral administration include pharmaceutically acceptable emulsions, microemulsions, solutions, suspensions, syrups, and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Some compositions of the invention relate to extended- or controlled-release formulations. These may be, for example, diffusion-controlled products, dissolution-controlled products, erosion products, osmotic pump systems or ionic resin systems. Diffusion-controlled products comprise a water-insoluble polymer which controls the flow of water and the subsequent egress of dissolved drug from the dosage from. Dissolution-controlled products control the rate of dissolution of the drug by using a polymer that slowly solubilizes or by microencapsulation of the drug—using varying thicknesses to control release. Erosion products control release of drug by the erosion rate of a carrier matrix. Osmotic pump systems release a drug based on the constant inflow of water across a semi permeable membrane into a reservoir which contains an osmotic agent. Ion exchange resins can be used to bind drugs such that, when ingested, the release of drug is determined by the ionic environment within the gastrointestinal tract.

Methods of Treatment

The invention contemplates treating diseases with an underlying proteinopathy. The proteinopathy may be described with reference to the underlying aggregating protein or with reference to one or more specific conditions that are associated with the aggregated protein. Commonly aggregated proteins include, amyloid beta, tau, Tar DNA Binding Protein 43 (TDP-43), Fused in sarcoma (FUS), α-synuclein, Huntingtin, Superoxide dismutase 1 (SOD-1), Prion proteins (PrP), mutant forms of Transthyretin, Atrophin 1 (ATN1; aka DRPLA), the Androgen receptor (AR), Ataxin 1 (ATXN1), Ataxin 2 (ATXN2), Ataxin 3 (ATXN3), Calcium Voltage-Gated Channel Subunit Alpha1 (ACACNA1A), Ataxin 7 (ATXN7), Protein Phosphatase 2 Regulatory Subunit Bbeta (PPP2R2B), and Tata Box Binding Protein (TBP). Accordingly, the invention contemplates treating conditions associated with aggregates of proteins selected from the group consisting of amyloid beta, tau, TDP-43, FUS, α-synuclein, Huntingtin, SOD-1, PrP, mutant forms of Transthyretin, Atrophin 1, the AR, Ataxin 1, Ataxin 2, Ataxin 3, ACACNA1A, Ataxin 7, PPP2R2B, and TBP.

Amyloid beta deposits are most prominently associated with Alzheimer's disease. Tau deposition is associated with many different conditions, including: Alzheimer's disease; Pick's Disease/Frontotemporal Dementia; Corticobasilar Degeneration; Progressive Supranuclear Palsy; Primary age-related tauopathy (PART)/Neurofibrillary tangle-predominant senile dementia; Infantile tauopathies (hemimegalencephaly (HME), tuberous sclerosis complex, focal cortical dysplasia type 2b (FCD2b), ganglioglioma); Lytico-bodig disease (Parkinson-dementia complex of Guam); ganglioglioma and gangliocytoma; meningioangiomatosis; postencephalitic parkinsonism; subacute sclerosing panencephalitis (SSPE); and argyrophilic grain disease (AGD). TDP-43 deposits are associated with: Lewy Body Disease; Frontotemporal Lobar Degeneration—TDP; limbic-predominant age-related TDP-43 encephalopathy (LATE); LATE-NC (neuropathological change); Amyotrophic lateral sclerosis; and multisystem proteinopathy. Dyanctin aggregation is associated with Perry Syndrome. FUS deposition is associated with Frontotemporal Lobar Degeneration—FUS and Amyotrophic lateral sclerosis. Deposition of α-synuclein is present in: multiple system atrophy; Parkinson's disease; Lewy Body Disease; and pure autonomic failure. Huntingtin aggregates are characteristic of Huntington's disease. Superoxide dismutase aggregation is present in some forms of Amyotrophic lateral sclerosis Prion proteins form insoluble aggregates in the spongiform encephalopathies. Transthyretin aggregates are found in: familial amyloidotic polyneuropathy; familial amyloidotic cardiomyopathy; familial amyloidotic poly-neuropathy (FAP); and senile systemic amyloidosis. Rhodopsin aggregation is a hallmark of retinitis pigmentosa. Valosin-containing protein forms aggregates in multisystem proteinopathy, which is also associated with mutations in prion-like domains of RNA binding proteins that also aggregate; and inclusion body myopathy (IBM) associated with Paget's disease of the bone (PDB), fronto-temporal dementia (FTD) and amyotrophic lateral sclerosis (ALS), which is sometimes called IBMPFD/ALS. Uromodulin deposits are present in UMOD-associated kidney disease. MUC-1 aggregates are found in MUC1-associated kidney disease. Aggregates of α1-antitrypsin and other proteins are found in respiratory epithelial cell proteinopathy (this is caused by surfactant protein A and C deficiencies). Immunoglobulin light-chain and immunoglobulin light-chain fragments are found aggregated in systemic amyloidosis as a complication of multiple myeloma. The foregoing disorders are all treatable according to the invention and other disorders caused or associated with the foregoing enumerated protein aggregates are also treatable according to the invention.

Specific disorders having underlying proteinopathies that are treatable according to the invention include Huntington's disease, autism spectrum disorder, Down syndrome (often associated with TDP-43), Alzheimer's Disease (AD), Dementia with Lewy Bodies (DLB), Frontotemporal Dementia (FTD, aka, Pick's Disease), and other forms of Frontotemporal Lobar Degeneration), head injuries, normal pressure hydrocephalus, Creutzfeldt-Jakob disease and other spongiform encephalopathies), amyotrophic lateral sclerosis, Corticobasilar Degeneration, Progressive Supranuclear Palsy, Frontotemporal Lobar Degeneration—TDP, limbic-predominany age-related TDP4-3 encephalopathy (LATE) and LATE-NC, Frontotemporal Lobar Degeneration—FUS, Multiple system atrophy, Familial amyloidotic polyneuropathy, Dentatorubropallidoluysian atrophy, Spinal and bulbar muscular atrophy, Spinocerebellar ataxias (including Types 1, 2, 3, 6, 7, 12 and 17), Machado-Joseph disease, pure autonomic failure, and Parkinson's disease among others.

In one prototypical proteinopathy, Huntington's disease (HD), the huntingtin protein forms aggregates, due to an amplification of a region encoding the amino acid glutamine. HD is an example of so-called poly-Q proteinopathies, due to the fact that that single letter code for glutamine is the letter Q. The following table provides a list of poly-Q disorders that are treatable according to the invention.

| Poly-Q Disorder | Affected Gene |
|---|---|
| DRPLA (Dentatorubropallidoluysian | Atrophin 1 (ATN1) aka DRPLA |
| atrophy) | |
| HD (Huntington's disease) | Huntintin (HTT) |
| SBMA (Spinal and bulbar muscular | Androgen receptor |
| atrophy) | |
| SCA1 (Spinocerebellar ataxia Type 1) | Ataxin 1 (ATXN1) |
| SCA2 (Spinocerebellar ataxia Type 2) | Ataxin 2 (ATXN2) |
| SCA3 (Spinocerebellar ataxia Type 3 or | Ataxin 3 (ATXN3) |
| Machado-Joseph disease) | |
| SCA6 (Spinocerebellar ataxia Type 6) | Calcium Voltage-Gated Channel |
| | Subunit Alpha1 (ACACNA1A) |
| SCA7 (Spinocerebellar ataxia Type 7) | Ataxin 7 (ATXN7) |
| SCA12 (Spinocerebellar ataxia Type | Protein Phosphatase 2 |
| 12) | Regulatory Subunit Bbeta |
| | (PPP2R2B) |
| SCA17 (Spinocerebellar ataxia Type | Tata Box Binding Protein (TBP) |
| 17) | |

Another prototypical proteinopathy is prion diseases in which the aggregates are made up of PrP, or more specifically PrPSc, which is the infectious form of the protein. The “Sc” in the name denotes scrapie, the prototypical prion disease identified in sheep. Human prion diseases treatable according to the invention include Creutzfeldt-Jakob disease (CJD), along with the CJD variants Iatrogenic Creutzfeldt-Jakob disease (iCJD), Variant Creutzfeldt-Jakob disease (vCJD), Familial Creutzfeldt-Jakob disease (fCJD), and Sporadic Creutzfeldt-Jakob disease (sCJD). Also included are Gerstmann-Sträussler-Scheinker syndrome (GSS), Fatal familial insomnia (FFI), Kuru, Familial spongiform encephalopathy and Variably protease-sensitive prionopathy (VPSPr) and fatal familial insomnia.

Another proteinopathy-associated condition, Chronic traumatic encephalopathy (CTE) is a neurodegenerative disease caused by repeated head injuries. CTE is a tauopathy characterized by the deposition of hyperphosphorylated tau (p-tau) protein as neurofibrillary tangles, astrocytic tangles and neurites in striking clusters around small blood vessels of the cortex. Severely affected cases show p-tau pathology throughout the brain. Abnormalities in phosphorylated 43 kDa TAR DNA-binding protein are found in most cases of CTE; beta-amyloid is identified in 43%, associated with age.

First-stage symptoms include attention deficit hyperactivity disorder as well as confusion, disorientation, dizziness, and headaches. Second-stage symptoms include memory loss, social instability, impulsive behavior, and poor judgment. Third and fourth stages include progressive dementia, movement disorders, hypomimia, speech impediments, sensory processing disorder, tremors, vertigo, deafness, depression and suicidality. Additional symptoms include dysarthria, dysphagia, cognitive disorders such as amnesia, and ocular abnormalities, such as ptosis. CTE can result in dementia.

The primary physical manifestations of CTE include a reduction in brain weight, associated with atrophy of the frontal and temporal cortices and medial temporal lobe. The lateral ventricles and the third ventricle are often enlarged, with rare instances of dilation of the fourth ventricle. As CTE progresses, there may be marked atrophy of the hippocampus, entorhinal cortex, and amygdala.

In one embodiment, the patient to be treated has a combination of proteinopathies. This patient typically presents with more severe forms of the associated disorders.

In one embodiment, said subject does not have protein aggregates comprising α-synuclein in their CNS. The most common proteinopathy-associated condition is dementia. Dementia is not itself a disease, but rather defines a set of symptoms related to a decline in memory and/or cognitive skills of such severity to adversely impact activities of daily living (Bruun 2018). Recognizing this, the definitive classification of dementia is based on the underlying neuropathology (Elahi 2017). With the exception of vascular dementia (VaD), dementia is considered a neurodegenerative disease.

The primary neurodegenerative dementias AD, DLB, Parkinson's Disease dementia, FTD, and dementia associated with prion diseases (like CJD) are characterized by progressive proteinopathy, which is an accumulation of misfolded proteins that lead to neuronal loss, neuroinflammation and glial reaction. Neurodegenerative dementias are differentiated by the location and nature of misfolded protein accumulation. Thus, an understanding of the applicable underlying pathology of the dementia is essential to inform rational treatment of what are considered different underlying conditions.

Shown below is a figure adapted from Briston and Hicks 2018, which shows the overlap between the proteinopathies and several prominent dementia-associated neurological disorders:

However, as AD, DLB progress, those patients also exhibit TDP43 accumulation.

Alzheimer's disease, for instance, is associated with amyloid plaques, consisting of aggregates of Abeta4, and fibrillary tangles, consisting of deposits of phosphorylated tau. Frontotemporal dementia is associated with deposits of tau, TDP-43 and/or FUS. On the other hand, pure vascular dementia is not associated with proteinopathy. Accordingly, Alzheimer's disease and frontotemporal dementia are included within the scope of the invention, but pure vascular dementia is not. The invention specifically contemplates treating patients with conditions associated with abnormal deposits of huntingtin protein, FUS, TDP-43, tau, amyloid-β (including Abeta42), optineurin, ubiquitin 2, superoxide dismutase 1, neurogenic locus notch homolog protein 3 (NOTCH3) and/or α-synuclein.

Diagnosis of proteinopathy-associated conditions can be done using imaging and measuring biomarkers in cerebrospinal fluid (CSF). The most widely used CSF biomarkers for Alzheimer's disease measure certain proteins: beta-amyloid 42 (the major component of amyloid plaques in the brain), tau, and phospho-tau (major components of tau tangles in the brain). In Alzheimer's disease, beta-amyloid 42 levels in CSF are low, and tau and phospho-tau levels are high, compared with levels in people without Alzheimer's or other causes of dementia. A putative biomarker for CTE is the presence in serum of autoantibodies against the brain. Brain autoantibodies may be produced due to a disrupted blood-brain barrier, exposing neuronal cells which are normally protected from the systemic immune system.

Imaging is as useful tool in diagnosing neurodegenerative proteinopathies, in particular computerized tomography (CT), magnetic resonance imaging (MRI) and positron emission spectroscopy (PET). Neural degeneration results in brain atrophy in CTE and this can be detected and quantified. Automated tools are increasingly available that can perform these functions.

Fluorodeoxyglucose (FDG) PET scans measure glucose use in the brain. Glucose, a type of sugar, is the primary source of energy for cells. Studies show that people with dementia often have abnormal patterns of decreased glucose use in specific areas of the brain. An FDG PET scan can show a pattern that may support a diagnosis of a specific cause of dementia.

Amyloid PET scans measure abnormal deposits of a protein called beta-amyloid. Higher levels of beta-amyloid are consistent with the presence of amyloid plaques, a hallmark of Alzheimer's disease. Several tracers may be used for amyloid PET scans, including florbetapir, flutemetamol, florbetaben, Pittsburgh compound B and NAV4694.

Tau PET scans detect abnormal accumulation of a protein, tau, which forms tangles in nerve cells in Alzheimer's disease and many other proteinopathies. Several tau tracers, such as AV-1451 (Flortaucipir), PI-2620, and MK-6240, are being studied in clinical trials and other research settings. One exemplary tracer is [18F]FDDNP, which is retained in the brain in individuals with a number of dementing disorders such as Alzheimer's disease, Down syndrome, progressive supranuclear palsy, corticobasal degeneration, familial frontotemporal dementia, and Creutzfeldt-Jakob disease. Other tau tracers include [18F]T807, [18F]T808, [18F]THK5117(5317), [18F]THK5351, [11C]PBB3, [18F]PM-PBB3, [18F]R069558948, and [18F]GTP1.

In accordance with the treatment methods of the present invention, administering a therapeutically effective amount of a ROCK inhibitor or a pharmaceutically acceptable salt thereof one or more times a day. The lowest therapeutically effective amount of fasudil, for example, is 70 mg per day, generally administered in 2 to 3 equal portions to obtain the full daily dose. The highest therapeutically effective dose may be determined empirically as the highest dose that remains effective in alleviating one or more dementia-related signs or symptoms, but does not induce an unacceptable level or adverse events. Fasudil, for example, generally will not be administered in a daily dose exceeding 180 mg. One preferred dosing regimen involves the treatment with 25, 30, 40 or 60 mg of Fasudil hydrochloride hemihydrate three times per day using an immediate-release formulation, for a total daily dose of 75-180 mg. Preferred dosing exceeds a daily dose of 70 mg, with most preferred ranges for daily dosing being 70 mg to 140 mg administered in three equal amounts during the day. Other preferred daily doses will range from 90 mg to 180 mg per day or 80 mg to 150 mg per day. A further dosing regimen involves the treatment with, 35 to 90 mg of Fasudil hydrochloride hemihydrate only two times per day using an immediate-release formulation, for a total daily dose of 70-180 mg. Generally, an oral daily dose of 70-75 mg will the minimum required to see a treatment effect. At more than 180 mg per day given orally, kidney function begins to be affected and higher dosing in most patients will not be warranted. Above 240 mg per day, kidney effects of the drug are generally unacceptable. Based on ROCK inhibitory activity, one skilled in the art can readily extrapolate the provided dosing ranges for fasudil to other ROCK inhibitors.

The treatment methods of the present invention, while contemplating various routes of administration, are particularly suited to oral administration. Thus, it will be understood that an effective amount of a ROCK inhibitor or a pharmaceutically acceptable salt thereof preferably is administered orally one or more times orally per day and an effective amount may range from the lowest therapeutically effective amount of fasudil, which is 70 mg per day. Generally, it will be administered orally in 2 to 3 equal portions to obtain the full daily dose. The daily oral dose of fasudil, for example, generally will not exceed 180 mg. One preferred dosing regimen involves the treatment with 25, 30, 40 or 60 mg of Fasudil hydrochloride hemihydrate three times per day orally using an immediate-release formulation, for a total daily dose of 75-180 mg. Preferred dosing exceeds a oral daily dose of 70 mg, with most preferred ranges for daily dosing being 70 mg to 140 mg administered in three equal amounts orally during the day. Other preferred daily doses will range from 90 mg to 180 mg per day or 80 mg to 150 mg orally per day. A further dosing regimen involves the treatment with, 35 to 90 mg of Fasudil hydrochloride hemihydrate only two times per day using an immediate-release oral formulation, for a total daily dose of 70-180 mg. Generally, an oral daily dose of 70-75 mg will the minimum required to see a treatment effect. At more than 180 mg per day given orally, kidney function begins to be affected and higher dosing in most patients will not be warranted. Above 240 mg per day orally, kidney effects of the drug are generally unacceptable. Based on ROCK inhibitory activity, one skilled in the art can readily extrapolate the provided dosing ranges for fasudil to other ROCK inhibitors.

In one embodiment, treatment of a proteinopathy patient with fasudil reduces or reverses the progression of dementia in the proteinopathy patient.

In a further embodiment, the reversal is measured by improvements in the MMSE score relative to the score before being treated with fasudil.

In another embodiment, treatment of a proteinopathy patient with fasudil reduces or reverses the loss of neural function comprises loss of cognitive function, autonomic function and/or motor function.

In a further embodiment, treatment of a proteinopathy patient with fasudil reduces neuroinflammation.

In another embodiment, the invention provides a method of reducing, reversing or preventing the accumulation of protein aggregates in tissue of a subject diagnosed as having a proteinopathy, or diagnosed as being at risk of developing a proteinopathy, the method comprising administering to said subject an effective amount of fasudil or a pharmaceutically acceptable salt thereof.

In one embodiment, the proteinopathy is a tauopathy. In a specific embodiment, the tauopathy is not also associated with an α-synucleinopathy. In a further embodiment, the tauopathy is primary age-related tauopathy (PART)/Neurofibrillary tangle-predominant senile dementia, characterized by the presence of tau NFTs similar to AD, but without amyloid beta plaques; Lytico-bodig disease (Parkinson-dementia complex of Guam); pure autonomic failure associated with alpha-synuclein or other proteinopathies, ganglioglioma and gangliocytoma; meningioangiomatosis; postencephalitic parkinsonism; subacute sclerosing panencephalitis (SSPE) and argyrophilic grain disease (AGD), hemimegalencephaly (HME), tuberous sclerosis complex, focal cortical dysplasia type 2b (FCD2b), and ganglioglioma, a tumor with dysplastic neurons and neoplastic glia cells.

In another embodiment, membrane-associated tau is reduced.

In another embodiment, the tauopathy is not associated with TDP43 or FUS.

In another specific embodiment, the proteinopathy is an α-synucleinopathy. In one embodiment, said svnucleinopathy is selected from Lewy Body Dementia, Parkinson's disease and multiple system atrophy.

In a further embodiment, the proteinopathy is associated with TDP43. In a specific embodiment, the proteinopathy is LATE and the patient is at least 80 years old.

In one embodiment, In embodiments, the protein associated with the proteinopathy is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

Certain patient sub-populations, such as renally impaired patients and/or older patients (e.g., 65 or older) may need lower doses or extended release formulations instead of immediate release formulations. Fasudil hydrochloride hemihydrate may have higher steady-state concentrations when given at usual doses to patients with renal disease and lower doses to lower the Cmax or delay the time to Cmax (increase the Tmax) may be required.

Renal dysfunction occurs with age and as the result of numerous disorders, including liver cirrhosis, chronic kidney disease, acute kidney injury (for example, due to administering a contrast agent), diabetes (Type 1 or Type 2), autoimmune diseases (such as lupus and IgA nephropathy), genetic diseases (such as polycystic kidney disease), nephrotic syndrome, urinary tract problems (from conditions such as enlarged prostate, kidney stones and some cancers), heart attack, illegal drug use and drug abuse, ischemic kidney conditions, urinary tract problems, high blood pressure, glomerulonephritis, interstitial nephritis, vesicoureteral, pyelonephritis, sepsis. Kidney dysfunction may occur in other diseases and syndromes, including non-kidney-related diseases that may occur along with kidney dysfunction, for example pulmonary artery hypertension, heart failure, and cardiomyopathies, among others.

Kidney function is most often assessed using serum (and/or urine) creatinine. Creatinine is a breakdown product of creatine phosphate in muscle cells and it is produced at a constant rate. It is excreted by the kidneys unchanged, principally through glomerular filtration. Accordingly, elevated serum creatinine is a marker for kidney dysfunction and it is used to estimate glomerular filtration rate.

Normal levels of creatinine in the blood are approximately 0.6 to 1.2 mg/dL in adult males and 0.5 to 1.1 mg/dL in adult females. When creatinine levels exceed these figures, the subject has renal dysfunction, and is, therefore, treatable according to the invention. Mild renal impairment/dysfunction occurs in the range of 1.2 mg/dL to 1.5 mg/dL. Moderate renal impairment/dysfunction is considered to occur at creatinine levels exceeding 1.5 mg/dL. Severe renal impairment, which includes what is considered to be renal failure, is defined as a serum creatinine level of ≥2.0 mg/dL or the use of renal replacement therapy (such as dialysis).

Treating subjects with mild, moderate and severe renal impairment is specifically contemplated.

As indicated, creatinine levels are considered to be a surrogate for glomerular filtration rate (GFR) and serum creatinine levels alone may be used to estimate glomerular filtration rate using the Cockroft-Gault equation.

According to the National Kidney Foundation, the following GFRs indicate the varying levels of renal function:

| GFR (ml/min/1.73 m²) | Renal Function |
|---|---|
| ≥90 | Normal or high |
| 60-89 | Mildly decreased |
| 45-59 | Mildly to moderately decreased |
| 30-44 | Moderately to severely decreased |
| 15-29 | Severely decreased |
| <15 | Kidney failure |

In general, creatinine clearance (estimated glomerular filtration rate) may be derived directly from serum creatinine using the Cockroft-Gault equation:

creatinine clearance=(((140−age in years)×(wt in kg))×1.23)/(serum creatinine in μmol/L)

For women the result of the calculation is multiplied by 0.85.

Empirically measured creatinine clearance may also be used directly as an estimate of glomerular filtration rate by looking at serum creatinine and urine creatinine levels. Specifically, urine is collected over 24 hours and the following equation is applied to ascertain creatinine clearance:

Creatinine Clearance (mL/min)=Urine Creatinine Concentration (mg/mL)*24 hour urine volume (mL)/Plasma Creatinine Concentration (mg/mL)*24 hour*60 minutes

In one embodiment, dose of fasudil for mild to moderate renal impairment is reduced to 50-80 mg per day. In another embodiment, the dose of fasudil is not reduced but is administered one time per day in an extended release dosage form.

In another embodiment, the dose is not reduced for mild to moderate renal impairment.

In one embodiment, the dose of fasudil is reduced to 30-45 for severe renal impairment. In another embodiment, the dose of fasudil is not reduced but is instead administered one time per day in an extended release dosage form.

In a further embodiment, the dose is reduced where serum creatinine (SCr)>2 and/or an increase in SCr>1.5× from baseline, and/or a decrease in eGFR>25% from baseline.

Patient size is an important factor to consider when using creatinine-based estimates of renal function. The units of drug clearance are volume/time (mL/min), whereas the units of estimated GFR for chronic renal disease are volume/time/standard size (mL/min/1.73 m²). Generally, doses may be adjusted down (e.g., 40-50 mg per day) for smaller patients and up for larger (e.g., 120 mg per day) for obese patients. A smaller male would be about 160 pounds or less. A smaller female patient would weigh about 130 pounds or less. Patients having a Body Mass Index of 30 and higher is considered obese.

In addition, older patients may need a lower dose at initiation, with a gradual increase to the recommended dose after days or weeks. In another embodiment, older patients may need lower doses for the duration of treatment. The aged population includes the “young old” who are 65-74, the “old old” who are 75-84 and the “frail elderly” who are 85 and older. For example, a starting dose of 30 mg per day for two weeks, followed by 60 mg per day for 4 weeks, then by 90 mg per day. Titration may even be warranted up to about 120 mg per day.

Another embodiment involves the treatment with 60-120 mg of fasudil hydrochloride hemihydrate once per day in an extended release dosage form. Treatment with an extended release total daily dose of 90 mg fasudil hydrochloride hemihydrate once per day is preferred. It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

Methods of administering compositions according to the invention would generally be continued for at least one day. Some preferred methods treat for up to 30 days or up to 60 days or even up to 90 days or even more. Treatment for more than 60 days is preferred and treatment for at least 6 months is particularly preferred. The precise duration of treatment will depend on the patient's condition and response to treatment. Most preferred methods contemplate that treatment begins after the onset or appearance of symptoms.

Another embodiment involves the treatment with 60-120 mg of Fasudil hydrochloride hemihydrate once per day in an extended release dosage form. Treatment with an extended release total daily dose of 90 mg Fasudil hydrochloride hemihydrate is preferred.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

Methods of administering compositions according to the invention would generally be continued for at least one day. Some preferred methods treat for up to 30 days or up to 60 days or even up to 90 days or even more. Treatment for more than 60 days is preferred and treatment for at least 6 months is particularly preferred. The precise duration of treatment will depend on the patient's condition and response to treatment.

Patients treatable according to the invention will typically score poorly on cognitive scales, such as the mini mental state exam (MMSE). A threshold of <23 on the MMSE is set for dementia, with score of ≤15 Representing severe dementia. Once the MMSE falls below 15, the Severe Impairment Battery (SIB) is a useful assessment too. Treatment using the inventive methods generally result in improved cognitive functioning. Patients will generally show improvement on the MMSE and the SIB of at least 3 points during the early stages of treatment and declines in cognition are slowed relative to control patients.

The MMSE, is described fully in Folstein (1975, 1987 and 2007). Generally, an MMSE score of 24-30 indicates no cognitive impairment, a score of 18-23 indicates mild cognitive impairment and 0-17 indicates severe cognitive impairment.

The methods of the invention also contemplate administering ROCK inhibitors with other compounds used to treat dementia or other symptoms of dementia. They may be administered in combination, a single dosage form, in a common dosing regimen or administered to the same patient at different times of the day using different dosing regiments.

In some embodiments, the patients are administered fasudil in combination with other actives approved to treat dementia, including but not limited to cholinesterase inhibitors and NMDA receptor antagonists. In one embodiment, the cholinesterase inhibitor is selected from the group consisting of donepezil, rivastigmine, and galantamine. Exemplary doses of the cholinesterase inhibitors include 3-25 mg per day, more preferably 6-12 mg per day. In another embodiment, the NMDA receptor antagonist is memantine. In a specific embodiment, memantine is administered at a dose of 5-28 mg per day, preferably 15-20 mg per day. In a further embodiment, the co-administered active is a combination of donepezil and memantine at a dose of 28 mg memantine and 10 mg donepezil.

In a specific embodiment, the combination of fasudil with cholinesterase inhibitors is administered to patients with proteinopathy-associated cortical dementia. In a further embodiment, the combination of fasudil with cholinesterase inhibitors is administered to patients with mixed dementia, especially in patients who have progressive proteinopathy.

Dextromethorphan hydrobromide is another an uncompetitive NMDA receptor antagonist that also has activity as a sigma-1 receptor agonist. Marketed in combination quinidine sulfate (a CYP450 2D6 inhibitor), the product Nudexta is indicated for the treatment of pseudobulbar affect, which occurs in many forms of dementia.

In a specific embodiment, the patient is administered fasudil in combination with cholinesterase inhibitors or NMDA antagonists has Alzheimer's dementia.

In a specific embodiment, the patient is administered fasudil in combination with cholinesterase inhibitors has Lewy Body dementia

In another embodiment, the patient is administered fasudil in combination with levodopa or a dopamine agonist, including but not limited to pramiprexole, ropinirole, apomorphine, and rotigotine. In a specific embodiment, the levodopa is administered in a dose of from about 30 to 2500 mg per day. In a further specific embodiment, the dopamine agonist is administered in a dose of from 0.25 to 10 mg per day. In another embodiment, fasudil is administered in combination with amantadine. In a specific embodiment, amantadine is administered in a dose of about 100-400 mg per day.

In yet another embodiment the patient is administered fasudil in combination with riluzole or edavarone at about 50 to 100 mg day.

In another embodiment, the patient is administered fasudil in combination with tetrabenzaine. In a specific embodiment, the tetrabenzaine is administered in a dose from 12.5 to 100 mg/patient/day.

In another embodiment, the patient is administered fasudil in combinantion with an anti-inflammatory.

In a further embodiment, the patient is administered fasudil in combination with an agent that enhances proteasome activity. Such agents inclue proflavine pimozide, cyclosporin A, mifepristone, chlorpromazine, loperamide, dipyrimidole, methylbenzethonium, verapamil, ursolic acid, betulinic acid, rolipram, DPCPX, PD169316, PAP1, PA26, PA28, TCH-165, MK-886, and AM-404.

In another embodiment, the patient is administered fasudil in combination with an agent that enhances autophagy. In one embodiment, the autophagy enhancer is BRD5631, carbamazepine, rapamycin, trehalose, trifluoperazine niguldipine, metformin, lithium carbonate, sodium valproate, and ABT-737.

In a further embodiment, the patient treated with fasudil is not also being treated with active agents including mood stabilizers, benzodiazepines, antipsychotics, anti-agitation drugs, or sleep aids. In a specific embodiment, the patient treated with fasudil is not being treated with risperidone, aripiprazole, quetiapine, carbamazepine, gabapentin, prazosin, trazodone or lorazepam.

In a further embodiment the patient treated with fasudil is being treated for depression. In a specific embodiment, the patient is treated with an anti-depressant such as citalopram or escitalopram.

In patients suffering from neuromuscular symptoms, functional rating scales are often applied. In ALS, for example, the ALS-Functional Rating Scale is often used. The Unified Huntington's Disease Rating Scale-Total Motor Score is often used in HD. Measures of functional independents and/or activities of daily living scales, like the Barthel Index, are often used to assess functional deficits. Any such scales may be used to assess levels of improvement (or reductions in decline) in patients treated according to the invention.

### LIST OF REFERENCES

Becker R E, Why do so many drugs for Alzheimer's disease fail in development? Time for new methods and new practices. J. Alzheimers Dis. 2008; 15(2): 303-25.
Briston T and Hicks A R, Mitochondrial dysfunction and neurodegenerative proteinopathies: mechanisms and prospects for therapeutic intervention. Biochem. Soc. Trans. 2018; 46(4): 829-42.
Bruun M et al., Evaluating combinations of diagnostic tests to discriminate different dementia types. Alzheimers & Dementia: Diagnosis, Assessment & Disease Monitoring. 2018; 10: 509-18.
Ceyzériat et al., Current Alzheimer Research 17: 1-13 (2020)
Chen M, Liu A, Ouyang Y, Huang Y, Chao X, Pi R. 2013. Fasudil and its analogs: a new powerful weapon in the long war against central nervous system disorders? Expert Opin Investig Drugs. 22:537-50.
Elahi F M and Miller B L, A clinicopathological approach to the diagnosis of demential. Nat. Rev. Neurol. 2017; 13(3): 457-76.
Elliott C, Rojo A, Ribe E, Broastock M, Xia W, Morin P, Semenov M, Baillie G, Cuadrado A, Al-Shawi R, Ballard C, Simons P, Killick R, A role for APP in Wnt signalling links synapse loss with β-amyloid production. Translational Psychiatry. 2018; 8(179).
Feng Y, LoGrasso P, Defert O, Li R, Rho Kinase (ROCK) Inhibitors and Their Therapeutic Potential. J Med Chem. 2016; 59*6): 2269-2300.
Folstein M F, Folstein S E, McHugh P R. “Mini-mental state”: a practical method for grading the cognitive state of patients for the clinician. J Psychiatr Res. 1975; 12:189-198.
Gurkar A. U. et al., Identification of ROCK1 kinase a a critica regulator of Beclin1-mediated autophagy during metabolic stress. Nat. Commun. 2013; 4:2189.
Hamano T, Shirafuji N; Yen S; Yoshida H, Kanaan N, Hayashi K, Ikawa M, Yamamura O, Fujita Y; Kuriyama M, Nakamoto Y, Rho-kinase ROCK inhibitors reduce oligomeric tau protein. Neurobiology of Aging; 2020; 89: 41-54.
Hou Y, Zhou L, Yang Q D, Du X P, Li M, Yuan M, Zhou Z W, Changes in hippocampal synapses and learning-memory abilities in a streptozotocin-treated rat model and intervention by using fasudil hydrochloride. Neuroscience. 2012; 200: 120-129.
Hu Y-B et al., Rock1 is Associated with Alzheimer's Disease-Specific Plaques, as well as Enhances Autophagosome Formation but not Autophagic ββ Clearance; Front. Cell. Neurosci. 2016; 10: 253. Jacobs M, Hayakawa K, Swenson L, Bellon S, Fleming M, Taslimi P, Doran J, The structure of dimeric ROCK I reveals the mechanism for ligand selectivity. J Biol Chem. 2006; 281(1): 260-68.
Nair A B and Jacob S, A Simple Practice Guide for Dose Conversion Between Animals and Human. J. Basic Clin Pharm. 2016; 7(2): 27-31.
Nakagawa O, Fukisawa K, Ishizaki T, Saito Y, Nakao K, Narumiya S, ROCK-I and ROCK-II, two isoforms of Rho-associated coiled-coil forming protein serine/threonine kinase in mice. FEBS Lett. 1996 Aug. 26; 392(2):189-93.
Peng H et al., CKD Stimulates Muscle Protein Loss Via Rho-Associated Protein Kinase 1 Activation. J. Am Soc Nephrol. 2016; 2792): 509-19.
Rovner B W, Folstein M F. Mini-mental state exam in clinical practice. Hosp Pract. 1987; 22(1A):99, 103, 106, 110.
Sasaguri H, Nilsson P, Hashimoto S, Nagata K, Saito T, De Strooper B, Hardy J, Vassar R, Winblad B, Saido T C, APP mouse models for Alzheimer's disease preclinical studies. EMBO J. 2017; 36(17): 2473-2487.
Sellers K, Elliott C, Jackson J, Ghosh A, Ribe E, Rojo A, Jarosz-Griffiths H H, Watson A A, Xia W, Semenov M, Morin P, Hooper N, Porter R, Preston J, Al-Shawi R, Baillie G, Lovestone S Cuadrado A, Harate M, Simons P, Srivastava D P, Killick R, Amyloid ß synaptotoxicity is Wnt-PCP dependent and blocked by fasudil. Alzeimer's & Dementia. 2018; 14: 306-317.
Shibuya M, Asano T, Sasaki Y. 2001. Effect of Fasudil HCl, a protein kinase inhibitor, on cerebral vasospasm. Acta Neurochir Suppl. 77:201-4.
Turk M. The Effect of Rho Kinase Inhibitors on Alzheimer's Disease, Dissertation. Arizona State University. May 2017.
Uehata M, Ishizaki T, Satoh H, Ono T, Kawahara T, Morishita T, Tamakawa H, Yamagami K, Maekawa M, Narumiya S, Calcium sensitization of smooth muscle mediated by a Rho-associated protein kinase in hypertension. Nature. 1997 Oct. 30; 389(6654):990-4.
Yamaguchi H, Miwa Y, Kasa M, Kitano K, Amano M, Kaibuchi K, Hakoshima T, Structural basis for induced-fit binding of Rho-kinase to the inhibitor Y-27632. J Biochem. 2006 September; 140(3):305-11.
Yu J, Gu Q, Yan Y, Yu H, Guo M, Liu C, Song G, Chai Z, Wang Q, Zia B, Zhang H, Jiang Y, Cungen M A, Fausidil improves cognition of APP/PS1 transgenic mice via inhibiting the activation of microglia and shifting microglia phenotypes from M1 to M2. Chin J Cell Mol Immunol. 2017; 33(12): 1585-1593.

The disclosure of each reference set forth herein is incorporated herein by reference in its entirety.

1. A method of treating a patient with a proteinopathy-associated condition, comprising administering a therapeutically effective amount of a rho kinase inhibitor to said patient, wherein the proteinopathy condition is selected from the group consisting of Huntington's disease, Down syndrome, normal pressure hydrocephalus, Creutzfeldt-Jakob disease and other spongiform encephalopathies), Corticobasilar Degeneration, Progressive Supranuclear Palsy, limbic-predominant age-related TDP4-3 encephalopathy (LATE) and LATE-NC, Frontotemporal Lobar Degeneration—TDP, Frontotemporal Lobar Degeneration—FUS, Multiple system atrophy, Familial amyloidotic polyneuropathy, Dentatorubropallidoluysian atrophy, Spinal and bulbar muscular atrophy, Spinocerebellar ataxias (including Types 1, 2, 3, 6, 7, 12 and 17), Machado-Joseph disease, pure autonomic failure, Parkinson's disease, multisystem proteinopathy retinitis pigmentosa, inclusion body myopathy (associated with Paget's disease of the bone, UMOD-associated kidney disease), MUC1-associated kidney disease, respiratory epithelial cell proteinopathy, familial amyloidotic cardiomyopathy, familial amyloidotic poly-neuropathy (FAP), senile systemic amyloidosis, and systemic amyloidosis as a complication of multiple myeloma.

2. The method according to claim 1, wherein the patient has Huntington's disease or Parkinson's disease.

3. The method according to claim 1 wherein the patient has dementia.

4. (canceled)

5. The method according to claim 3 wherein the patient does not have vascular dementia.

6. The method according to claim 1 wherein the treatment results in a greater-than 3-point improvement on the mini mental state exam.

7. The method according to claim 1 wherein the rho kinase inhibitor is an isoquinoline derivative.

8. The method according to claim 7 wherein the isoquinoline derivative is fasudil, a salt, or a derivative thereof.

9. The method according to claim 7 wherein said derivative is M3.

10. The method according to claim 1 where said treatment continues for at least 6 months.

11. The method according to claim 7, wherein said isoquinoline derivative is administered in a dose of at least 70 mg per day.

12. The method according to claim 11, wherein said dose is administered in three equal portions throughout the day.

13. The method according to claim 11, wherein the total daily dose is between 70 mg and 180 mg

14. The method according to claim 13, wherein the total daily dose exceeds 70 mg and is administered in a sustained release formulation.

15. The method according to claim 1, wherein said patient has a proteinopathy associated with Parkinson's disease or Down syndrome.

16. (canceled)

17. The method according to claim 1, wherein the proteinopathy is characterized by deposits containing one or more of the following in normal or mutant form: amyloid beta, tau, Tar DNA Binding Protein 43 (TDP-43), Fused in sarcoma (FUS), α-synuclein, Huntingtin, Superoxide dismutase 1 (SOD-1), Prion proteins (PrP), mutant forms of Transthyretin (TTR), Atrophin 1 (ATN1), the Androgen receptor (AR), Ataxin 1 (ATXN1), Ataxin 2 (ATXN2), Ataxin 3 (ATXN3), Calcium Voltage-Gated Channel Subunit Alpha1 (ACACNA1A), Ataxin 7 (ATXN7), Protein Phosphatase 2 Regulatory Subunit Bbeta (PPP2R2B), dynactin, rhodopsin, valosin-containing protein, uromodulin, MUC-1, α1-antitrypsin, immunoglobulin light chain, an immunoglobulin light chain fragment and Tata Box Binding Protein (TBP).

18. The method according to claim 17 wherein the proteinopathy is characterized by deposits comprising α-synuclein.

19. The method according to claim 18 wherein the patient has multiple system atrophy.
